(19) United States
(12) Patent Application Publication
(10) Pub. No.: US 2024/0279360 A1
(43) Pub. Date: Aug. 22, 2024

(54) BIFUNCTIONAL COMPOUNDS CONTAINING IGF-2 POLYPEPTIDES

(71) Applicant: LYCIA THERAPEUTICS, INC., South San Francisco, CA (US)

(72) Inventors: Tigran Arvid AIVAZIAN, South San Francisco, CA (US); James Richard GLYNNE, South San Francisco, CA (US); Dallas Clifford JONES, South San Francisco, CA (US); Stephanie Ann PINKERTON, South San Francisco, CA (US); Darrin Anthony LINDHOUT, South San Francisco, CA (US); Sarah M. MCWHIRTER, South San Francisco, CA (US); Jason G. LEWIS, South San Francisco, CA (US)

(21) Appl. No.: 18/568,767

(22) PCT Filed: Jun. 23, 2022
(86) PCT No.: PCT/US2022/034777

§ 371 Date: Dec. 8, 2023

Related U.S. Application Data
(60) Provisional application No. 63/214,004, filed on Jun. 23, 2021.

(51) Int. Cl. C07K 16/42; A61K 47/68; C07K 14/65

(52) U.S. Cl. CPC C07K 16/4291; A61K 47/6873; C07K 14/65; C07K 2317/77; C07K 2317/92; C07K 2319/30; C07K 2319/33

(57) ABSTRACT

The present disclosure provides a class of bifunctional compounds that includes an IGF-2 polypeptide that specifically binds to a cell surface cation independent mannose-6-phosphate receptor (CI-M6PR), and a target binding moiety. The bifunctional compounds can trigger the CI-M6PR cell surface receptor to internalize into the cell a complex of the target and the bifunctional compound. The target can be an extracellular target protein such as a soluble protein or a membrane bound target protein. The target binding moiety can be an antibody or antibody fragment. Also provided are methods of using the bifunctional compounds for sequestration and/or lysosomal degradation of a target, e.g., an extracellular target protein associated with a disease or disorder of interest.

### 1. CROSS REFERENCE TO RELATED APPLICATIONS

This application claims priority to U.S. Provisional Application No. 63/214,004, filed Jun. 23, 2021, which is hereby incorporated by reference in its entirety.

### 2. SEQUENCE LISTING

The instant application contains a Sequence Listing which has been submitted via EFS-Web and is hereby incorporated by reference in its entirety. Said ASCII copy, created on Month XX, 2022, is named 47964WO_sequencelisting.txt and is X,XXX,XXX bytes in size.

### 3. INTRODUCTION

### 3.1. Field of the Invention

This disclosure provides bifunctional molecules having an insulin-like growth factor 2 (IGF-2) polypeptide and a target-binding moiety. The IGF-2 polypeptide can specifically bind to, e.g., cation independent mannose-6-phosphate receptors (CI-M6PR) on the surface of cells of interest to trigger internalization of the bifunctional molecule and a bound target moiety of interest.

### 3.2. Description of Related Art

Insulin-like growth factor-2/cation independent mannose-6-phosphate receptor (IGF2R/CI-M6PR) is a single trans-membrane glycoprotein that is widely but selectively distributed. IGF2R/CI-M6PR is involved in the trafficking of mannose-6-phosphate (M6P)-containing lysosomal enzymes from the trans-Golgi network to the endosomes and lysosomes.

Lysosomal storage disorders (LSDs) are a class of approximately 50 different human metabolic diseases caused by a deficiency for specific lysosomal proteins that results in the accumulation of various substances within the endosomal/lysosomal compartments.

Insulin-like growth factor 2 (IGF-2) is a protein hormone structurally related to insulin, and having growth-regulating, insulin-like and mitogenic activities. IGF-2 exerts its effects by binding to the IGF-1 receptor and to isoforms of the insulin receptor (e.g., IR-A). IGF-2 also binds to the IGF-2 receptor (also referred to as the cation-independent mannose 6-phosphate receptor (CI-M6PR), which can act as an IGF-2 signaling antagonist.

Variants of IGF-2 peptides have been directly conjugated or fused with recombinant lysosomal enzymes for intracellular delivery of the enzymes in enzyme replacement therapy for lysosomal storage disorders.

### 4. SUMMARY OF THE INVENTION

The present disclosure provides a class of bifunctional compounds that includes an IGF2 polypeptide that specifically binds to a cell surface receptor (e.g., mannose-6-phosphate receptor (M6PR)), and a target binding moiety. The bifunctional compounds can trigger the cell surface receptor (e.g., M6PR) to internalize into the cell a complex of the bifunctional compound and a bound target. The target can be an extracellular target protein such as a soluble protein or a membrane bound target protein. The target binding moiety can be an antibody or antibody fragment. Also provided are methods of using the bifunctional compounds for sequestration and/or lysosomal degradation of a target, e.g., an extracellular target protein associated with a disease or disorder of interest.

### 5. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 shows a schematic of an exemplary bifunctional compound of this disclosure that includes a target-binding antibody linked to two IGF-2 polypeptides. Site specific covalent linkages to the antibody can be achieved via conjugation of a chemoselective bivalent linker, such as 6-maleimidocaproic acid sulfo-NHS. The linker can be installed on an IGF-2 polypeptide via e.g., NHS chemistry and coupling of the linker to an N-terminal amino group or sidechain lysine group of the IGF-2 polypeptide. The maleimide chemoselective ligation group of the linker shown in FIG. 1 is reactive with cysteine residues on the antibody, such as a cysteine residue engineered into the antibody at desirable site-specific locations, e.g., L443C.

FIG. 2 shows a schematic of an exemplary bifunctional compound that includes a target-binding antibody fused to four IGF-2 polypeptides at the C-terminals of the immunoglobulin heavy and light chains of the antibody. The bifunctional compound can be a fusion protein where IGF-2 polypeptides are incorporated into the architecture of a target-binding antibody at a variety of suitable sites.

FIGS. 3A-3C shows uptake of exemplary bifunctional compound (1) IGF-2-omalizumab (designed “IGF2” in the graph legend) in three different cell types. FIG. 3A shows uptake in human Jurkat cells. FIG. 3B shows uptake in mouse C2C12 cells. FIG. 3C shows uptake in mouse fibroblasts. The cellular uptake is compared to two different omalizumab conjugates having glycan ligands for M6PR (i.e., a linked mannose-6-phosphate glycan conjugate designated “M6P”, or to unconjugated omalizumab (i.e., “UNLB”). Each of the omalizumab compositions tested was fluorescently labelled with an Alexa 488 fluorophore reagent dye to provide for assessment of cellular uptake via mean fluorescent intensity (MFI) of cells using flow cytometry.

FIG. 4 shows the results of a cellular uptake assay illustrating that exemplary bifunctional compound (1) IGF-2-omalizumab (“IGF2”) is internalized into wild type K562 cells having M6PR but not into K562 M6PR-knockout (KO) cells. Similar results were observed for omalizumab conjugates with the glycan ligands for M6PR (“M6P” or “M6Pn”). “UNLB” is the control unconjugated omalizumab. Cellular uptake was assessed using flow cytometry to determine mean fluorescent intensity (MFI) of cells.

### 6. DETAILED DESCRIPTION OF THE INVENTION

### 6.1. Bifunctional Compounds

Aspects of this disclosure include a bifunctional compound that includes an IGF-2 polypeptide connected to a target-binding moiety. Interaction of the IGF-2 polypeptide with a cell surface receptor (e.g., M6PR) can trigger internalization of the bifunctional compound in complex with the target moiety, via receptor-mediated endocytosis. In some embodiments, the target moiety is a target protein and the bifunctional compound is capable of facilitating internalization, followed by degradation, of the target protein in the lysosome.

### 6.1.1. IGF-2 Polypeptides

Insulin-like growth factor-2 (IGF-2) is a protein hormone encoded by the IGF2 gene and having growth-regulating, insulin-like and mitogenic activity. The terms “IGF-2 polypeptide”, “IGF-2 protein” and “IGF-2 peptide” are used interchangeably herein and refer to polypeptides that include an amino acid sequence corresponding to a naturally occurring IGF-2 protein hormone, variants thereof, truncated versions thereof, and/or a fragment thereof. In general, the IGF-2 polypeptides selected for incorporation into the bifunctional compounds or conjugates of this disclosure are polypeptides capable of binding to a cell surface receptor and to trigger receptor-mediated endocytosis, thereby facilitating uptake of the bifunctional molecule and a bound target moiety into a lysosome in a cell.

The term “mannose-6-phosphate receptor” and “M6PR” refer to receptors of the family of mannose-6-phosphate receptors. M6PRs are transmembrane glycoprotein receptors that target enzymes to lysosomes in cells. MP6R endogenously transports proteins bearing N-glycans capped with mannose-6-phosphate (M6P) residues to lysosomes, and cycles between endosomes, the cell surface, and the Golgi complex. See, e.g., Ghosh et al., Nat. Rev. Mol. Cell Biol. 2003; 4: 202-213. The family of M6PRs includes the cation independent mannose-6-phosphate receptor (CI-M6PR). The CI-M6PR is also referred to as the insulin-like growth factor 2 receptor (IGF2R) and is encoded in humans by the IGF2R gene (see NCBI Gene ID: 3482). The CI-M6PR binds insulin-like growth factor 2 (IGF-2) and mannose-6-phosphate (M6P)-tagged proteins.

Naturally occurring human IGF-2 binds to a number of cell surface receptors with varying affinity, such as IGFR1, insulin receptor, and cation-independent mannose-6-phosphate receptor (CI-M6PR). IGF-2 can exert its biological effect primarily through interactions with the IGF1R and insulin receptor while interaction with the CI-M6PR is believed to result in the IGF-2 being internalized to the lysosome where it is degraded.

In some embodiments, an IGF-2 polypeptide suitable for incorporation in the bifunctional compounds of this disclosure is one that binds specifically to the CI-M6PR. Particularly useful are mutations, variations and/or truncations in the IGF-2 polypeptide that result in a variant polypeptide which binds the CI-M6PR with a substantially equivalent or higher affinity, while binding other receptors of interest with reduced affinity, relative to a naturally occurring parental or wild type IGF-2 polypeptide. In some embodiments, the IGF-2 polypeptide is a variant IGF-2 polypeptide having enhanced affinity for the CI-M6PR as compared to naturally occurring human IGF-2 polypeptide.

In some embodiments, the IGF-2 polypeptide is a variant IGF-2 polypeptide that has diminished or decreased, or no affinity for the insulin receptor and/or IGF-1 receptor (IGF1R) as compared to a naturally occurring parental IGF-2 polypeptide. In some embodiments, the IGF-2 peptide polypeptide is a variant having increased affinity for the CI-M6PR as compared to a naturally occurring parental or wild type IGF-2 polypeptide. In some embodiments, the IGF-2 polypeptide has mutations or variations that result in a polypeptide which binds the CI-M6PR with high affinity while no longer binding the other two receptors (insulin receptor and/or IGF1R) with appreciable affinity. In some embodiments, the IGF-2 polypeptide includes a substitution of residues Tyr 27 with Leu, Leu 43 with Val, and/or Ser 26 with Phe which diminishes the affinity of the resulting IGF-2 polypeptide for IGF1R (see e.g., Tones et al. (1995) J. Mol. Biol. 248(2):385-401).

In some embodiments, the IGF-2 polypeptide is a truncated polypeptide missing residues 1-7 of wild type IGF-2 (e.g., mature human IGF-2) which results in a relative decrease in affinity for the IGF1R (see e.g., Hashimoto et al. (1995) J. Biol. Chem. 270(30): 18013-8). In some embodiments, the IGF-2 polypeptide is a truncated polypeptide missing residues 2-7 of wild type IGF-2 (e.g., mature human IGF-2). In some embodiments, the IGF-2 polypeptide is a C-terminal truncated polypeptide, e.g., a polypeptide missing the residues 62-67 of wild type IGF-2, which results in a lower affinity for the resulting IGF-2 polypeptide for IGF1R (see e.g., Roth et al. (1991) Biochem. Biophys. Res. Commun. 181(2):907-14).

In some embodiments, an IGF-2 polypeptide further contains a deletion or a replacement of amino acids corresponding to positions 2-7 of SEQ ID NO:1. In some embodiments, an IGF-2 polypeptide further includes a deletion or a replacement of amino acids corresponding to positions 1-7 of SEQ ID NO: 1. In some embodiments, an IGF-2 polypeptide further contains a deletion or a replacement of amino acids corresponding to positions 62-67 of SEQ ID NO: 1. In some embodiments, an IGF-2 polypeptide further contains an amino acid substitution at a position corresponding to Tyr27, Leu43, or Ser26 of SEQ ID NO:1. In some embodiments, an IGF-2 polypeptide contains at least an amino acid substitution selected from the group consisting of Tyr27Leu, Leu43 Val, Ser26Phe and combinations thereof. In some embodiments, an IGF-2 polypeptide contains amino acids corresponding to positions 48-55 of SEQ ID NO: 1. In some embodiments, an IGF-2 polypeptide contains at least three amino acids selected from the group consisting of amino acids corresponding to positions 8, 48, 49, 50, 54, and 55 of SEQ ID NO: 1. In some embodiments, an IGF-2 polypeptide contains, at positions corresponding to positions 54 and 55 of SEQ ID NO: 1, amino acids each of which is uncharged or negatively charged at pH 7.4. In some embodiments, the IGF-2 polypeptide has diminished binding affinity for the IGF-1 receptor (IGFR1) relative to the affinity of naturally-occurring human IGF-2 for the IGF-1 receptor.

In some embodiments, the IGF-2 polypeptide is a variant IGF-2 polypeptide having diminished or no affinity for the insulin receptor and/or IGFR1 as compared to naturally occurring human IGF-2 polypeptide.

In some embodiments, the IGF-2 polypeptide is an active fragment of a wild type IGF-2 (e.g., mature human IGF-2). In some embodiments, the IGF-2 polypeptide is an active fragment having a sequence of 30 amino residues or less, such as 20 amino acid residues or less, 15 amino residues or less, 12 amino residues or less, or even 10 amino residues or less. In some embodiments, the IGF-2 polypeptide is an active fragment that includes residues 12-20, such as residues 12-20 of a wild type IGF-2 (e.g., mature human IGF-2), or a variant thereof. In some embodiments, the IGF-2 polypeptide is linked to the bifunctional compound via a spacer polypeptide (e.g., as described herein).

In some embodiments, the IGF-2 polypeptide is a variant modified to minimize binding to serum IGF-binding proteins (see e.g., Baxter (2000) Am. J. Physiol Endocrinol Metab. 278(6):967-76) to avoid sequestration of the bifunctional compounds in vivo. The IGF-2 polypeptide can be a variant where amino acid residues necessary for binding of IGF-2 polypeptides to IGF-binding serum proteins in vivo are replaced with variant residues that provide for reduced affinity for the IGF-binding serum proteins while retaining high affinity binding to CI-M6PR. In some embodiments, the IGF-2 polypeptide is a variant including replacement of Phe-26 with Ser (see e.g., Bach et al. (1993) J. Biol. Chem. 268(13):9246-54), and/or replacement of Glu-9 with Lys.

Accordingly, the compounds of this disclosure can specifically bind to an internalizing CI-M6PR cell surface receptor via binding of the IGF-2 polypeptide(s). In particular embodiments, the surface M6PR is a human M6PR.

In some embodiments, the variant IGF-2 polypeptide includes a replacement of Phe 26 of IGF-2 with Ser that provides for reduced affinity of the resulting variant IGF-2 polypeptide for serum IGFBP-1 and -6 with no effect on binding to the M6P/IGF-2 receptor. In some embodiments, the variant IGF-2 polypeptide includes other substitutions, such as Ser for Phe 19 and/or Lys for Glu 9.

In some embodiments, the bifunctional compound is a fusion of the IGF-2 polypeptide and a target-binding polypeptide, e.g., a peptide, protein, or antibody or antibody fragment that specifically binds the target moiety, and the IGF-2 polypeptide is a IGF2 polypeptide variant that confers improved expression and/or secretion of a fusion protein bifunctional compound, compared to a naturally occurring IGF-2 polypeptide. In some embodiments, the IGF-2 polypeptide is a furin-resistant variant IGF-2 polypeptide having an amino acid sequence at least 70% identical to a IGF-2 polypeptide sequence of Table 1, and a mutation that abolishes at least one furin protease cleavage site. Furin-resistant IGF-2 polypeptides of interest include those described in U.S. Pat. No. 9,469,683.

In some embodiments, the IGF-2 polypeptide is a variant that includes amino acids 8-67 of mature human IGF-2 polypeptide. In some embodiments, the IGF-2 polypeptide is a variant that includes an Ala substitution at position Arg37 (e.g., SEQ ID NO: 6), where the IGF-2 polypeptide (i) has diminished binding affinity for the insulin receptor relative to the affinity of naturally-occurring human IGF-2 polypeptide for the insulin receptor, (ii) is resistant to furin cleavage and (iii) binds to the human cation-independent mannose-6-phosphate receptor in a mannose-6-phosphate-independent manner.

In some embodiments, the IGF-2 polypeptide is a variant that includes SEQ ID NO:3. In some embodiments, the IGF-2 polypeptide is a variant that includes one or more of the following modifications with respect to a parent IGF-2 sequence, e.g., of Table 1:
substitution of arginine for glutamic acid at position 6;
deletion of amino acids 1-4 and 6;
deletion of amino acids 1-4, 6 and 7;
deletion of amino acids 1-4 and 6 and substitution of lysine for threonine at position 7;
deletion of amino acids 1-4 and substitution of glycine for glutamic acid at position 6 and substitution of lysine for threonine at position 7;
substitution of leucine for tyrosine at position 27; and
substitution of leucine for valine at position 43.

IGF-2 polypeptides of interest include those described in WO2005/078077, WO2012166653, WO2014/085621, U.S. Pat. Nos. 9,469,683, 10,301,369, 10,660,972 and WO2021/072372, the disclosures of which are incorporated herein by reference in their entirety. The sequences of exemplary IGF-2 polypeptides of interest are shown in Table 1. In various embodiments, any one or more of the sequence variations, mutations and/or truncations described herein as imparting desirable properties on the IGF-2 polypeptides can be applied to a parental IGF-2 polypeptide sequence (e.g., a sequence of Table 1) to produce a variant IGF-2 polypeptide of interest. All such variant IGF-2 polypeptide sequences are meant to be encompassed by this disclosure.

TABLE 1
IGF-2 polypeptide sequences of interest

| SEQ | | |
|---|---|---|
| ID | | |
| NO: | Name | Sequence |
| 1 | mature human | AYRPSETLCGGELVDTLQFVCGDRGFYFSRPASRVSR |
| | IGF-2 | RSRGIVEECCFRSCDLALLETYCATPAKSE |
| 2 | Δ2-7 IGF-2 | AALCGGELVDTLQFVCGDRGFYFSRPASRVSRRSRGI |
| | | VEECCFRSCDLALLETYCATPAKSE |
| 3 | Δ1-4 IGF-2 | SRTLCGGELVDTLQPVCGDRGFLFSRPASRVSRRSRGI |
| | | VEECCFRSCDLALLETYCATPARSE |
| 4 | 7-67 IGF-2 | TLCGGELVDTLQFVCGDRGFYFSRPASRVSRRSRGIVE |
| | | ECCFRSCDLALLETYCATPAKSE |
| 5 | 8-67 IGF-2 | LCGGELVDTLQFVCGDRGFYFSRPASRVSRRSRGIVEE |
| | | CCFRSCDLALLETYCATPAKSE |
| 6 | 8-67 IGF-2 | LCGGELVDTLQFVCGDRGFYFSRPASRVSARSRGIVEE |
| | (R37A) | CCFRSCDLALLETYCATPAKSE |
| 7 | 12-21 IGF-2 | ELVDTLQFVS |
| | (C21S) (Tc | |
| | peptide) | |
| 8 | 12-21 IGF-2 | ELVDWLQFVS |
| | (T16W, C21S) | |
| | (Tl peptide) | |
| 9 | 12-21 IGF-2 | ELVDYLQFVS |
| | (T16Y, C21S) | |
| | (T2 peptide) | |
| 10 | 12-21 IGF-2 | ELVDTLQFVW |
| | (C21W) (T3 | |
| | peptide) | |
| 11 | 12-21 IGF-2 | ELVDFLQFVS |
| | (T16F, C21S) | |
| | (T4 peptide) | |
| 12 | 12-21 IGF-2 | ELVDTLQFVR |
| | (C21R) (T5 | |
| | peptide) | |
| 13 | (P431 peptide) | VHWDFRQWWQPS |

In some embodiments, the IGF-2 polypeptide has an amino acid sequence that is at least 80, 85, 90, 95, 96, 97, 98 or 99% identical to an IGF2 variant peptide of Table 1. In some embodiments, the IGF-2 polypeptide comprises an amino acid sequence that is at least 90, 95, or 98% identical to an IGF2 variant peptide selected from SEQ ID NO: 1-6 of Table 1. In some embodiments, the IGF-2 polypeptide comprises an amino acid sequence that is at least 80%, or 90% identical to an IGF2 variant peptide selected from SEQ ID NO: 7-13 of Table 1.

### 6.1.2. Targets

As summarized above, the bifunctional compounds of this disclosure can bind specific targets of interest and provide for their removal from the cell surface or from the extracellular milieu. For example, the bifunctional compounds may provide for cell surface receptor (e.g., CI-M6PR)-mediated internalization and/or degradation of a target molecule of interest in a cell's lysosome.

In some embodiments, target protein is one that is associated with a disease or disorder of interest. Diseases and disorders of interest include where a desirable treatment could include depletion of the certain target proteins. Target proteins of interest include, for example, soluble proteins, e.g., secreted proteins, cell surface proteins (for example, cell surface receptor proteins, e.g., tyrosine kinase receptors, soluble cytokine receptors, and immune checkpoint receptors, e.g., EGFR, VEGFR, FGFR, and PD-L1), lectins, complements, lipoproteins, transport proteins, MHC class I and class II molecules, cytokines, chemokines, and/or receptors, or fragments or subunits of any of the foregoing.

In some embodiments, the target protein is a membrane bound protein. In some embodiments, the target protein is a soluble extracellular protein. In some embodiments, the target protein is associated with a disease or disorder of interest.

In certain embodiments, the target protein is a VEGF protein.

In certain embodiments, the target protein is a TNF protein (e.g., TNF-alpha).

In certain embodiments, the target protein is a PD-L1 protein.

In certain embodiments, the target protein is an EGFR protein, a VEGFR protein, a FGFR2 protein or a FGFR3 protein.

### 6.1.3. Target-Binding Moieties

The target-binding moiety can be any moiety that has an affinity for the target of less than 1 μM, such as 300 nM or less, 100 nM or less, 30 nM or less, 10 nM or less, 3 nM or less, or 1 nM or less, e.g., as measured in an in vitro binding assay.

In some embodiments, the target-binding moiety is a biomolecule. In some embodiments, the target-binding moiety is a biomolecule that specifically binds to a target protein. In some embodiments, the biomolecule is selected from peptide, protein, polynucleotide, polysaccharide, glycan, glycoprotein, lipid, enzyme, antibody, and antibody fragment.

In some embodiments, the target-binding moiety is a polypeptide (e.g., peptide or protein binding motif, protein domain, engineered polypeptide, or glycoprotein) that specifically binds to a target molecule, such as a target protein. In some embodiments, the target-binding moiety of the bifunctional compound includes a polypeptide that binds to a soluble (e.g., secreted) target protein of interest. In some embodiments, the target-binding is a polypeptide ligand that includes a receptor ligand, or a receptor-binding portion or fragment of the receptor ligand, that binds a target cell surface receptor. Target-binding polypeptides may contain L-amino acids, D-amino acids, or both and may contain any of a variety of naturally occurring amino acids, non-naturally occurring amino acids, and/or amino acid modifications or analogs known in the art. Useful modifications include, e.g., N-terminal acetylation, amidation, methylation, etc.

In some embodiments, the target-binding moiety is a polynucleotide that specifically binds to a target molecule, such as a target protein or a target nucleic acid. The terms polynucleotide and nucleic acid can be used interchangeably. In some embodiments, the target-binding moiety is a nucleic acid aptamer that specifically binds to a target molecule, such as a target protein.

In some embodiments, the target-binding moiety is a glycan. In some embodiments, the target-binding moiety is a glycan epitope for an autoantibody.

### 6.1.3.1 Antibodies

In some embodiments, the target-binding moiety is an antibody or antibody fragment that specifically binds to a target moiety, such as a target protein.

The IGF-2 polypeptide can be site-specifically covalently linked to the antibody or antibody fragment, via an optional linking moiety. IGF-2 polypeptide can be covalently linked to the antibody or antibody fragment via a site-specific cysteine modification on the antibody or antibody fragment (e.g., L443C) and a thiol-reactive chemoselective ligation group. IGF-2 polypeptide can be covalently linked to the antibody or antibody fragment via one or more lysine residues of the antibody or antibody fragment and an amine-reactive chemoselective ligation group.

The IGF-2 polypeptide can be linked to the target-binding antibody or antibody fragment via a chimeric protein fusion, via an optional spacer sequence.

In some embodiments, the bifunctional compound of this disclosure includes an antibody (Ab). In some embodiments, Ab is a monoclonal antibody. In some embodiments, Ab is a human antibody. In some embodiments, Ab is a humanized antibody. In some embodiments, Ab is a chimeric antibody. In some embodiments, Ab is a full-length antibody that includes two heavy chains and two light chains. In some embodiments, Ab is an IgG antibody, e.g., is an IgG1, IgG2, IgG3 or IgG4 antibody. In some embodiments, Ab is a single chain antibody. In some embodiments, the target-binding moiety is an antigen-binding fragment of an antibody, e.g., a Fab fragment.

In some embodiments, the antibody or antibody fragment specifically binds to a cancer antigen.

In some embodiments, the antibody or antibody fragment specifically binds to a hepatocyte antigen.

In some embodiments, the antibody or antibody fragment specifically binds to an antigen presented on a macrophage.

In some embodiments, the antibody or antibody fragment specifically binds to an intact complement or a fragment thereof. In some embodiments, the antibody or antibody fragment specifically binds to one or more immunodominant epitope(s) within intact complement or a fragment thereof.

In some embodiments, the antibody or antibody fragment specifically binds to a cell surface receptor. In some embodiments, the antibody or antibody fragment specifically binds to a cell surface receptor ligand.

In some embodiments, the antibody or antibody fragment specifically binds to an epidermal growth factor (EGF) protein, e.g., a human EGF. In some embodiments, the antibody or antibody fragment specifically binds to one or more immunodominant epitope(s) within an EGF protein.

In some embodiments, the antibody or antibody fragment specifically binds to an epidermal growth factor receptor (EGFR) protein, e.g., a human EGFR. In some embodiments, the antibody or antibody fragment specifically binds to one or more immunodominant epitope(s) within an EGFR protein. In some embodiments, the antibody or antibody fragment comprises the CDRs present in cetuximab. In some embodiments, the antibody or antibody fragment includes the variable light chain and variable heavy chain present in cetuximab. In some embodiments, the antibody is cetuximab. In some embodiments, the antibody or antibody fragment includes the CDRs present in matuzumab. In some embodiments, the antibody or antibody fragment includes the variable light chain and variable heavy chain present in matuzumab. In some embodiments, the antibody is matuzumab.

In some embodiments, the antibody or antibody fragment specifically binds to vascular endothelial growth factor (VEGF) protein, e.g., human VEGF protein. In some embodiments, the antibody or antibody fragment specifically binds to one or more immunodominant epitope(s) within a VEGF protein.

In some embodiments, the antibody or antibody fragment specifically binds to a vascular endothelial growth factor receptor (VEGFR) protein, e.g., human VEGFR protein. In some embodiments, the antibody or antibody fragment specifically binds vascular endothelial growth factor receptor 2 (VEGFR2) protein, e.g., a human VEGFR2 protein. In some embodiments, the antibody or antibody fragment specifically binds a vascular endothelial growth factor receptor 3 (VEGFR3) protein, e.g., a human VEGFR3 protein. In some embodiments, the antibody or antibody fragment specifically binds to one or more immunodominant epitope(s) within a VEGFR protein, a VEGFR2 protein or a VEGFR3 protein.

In some embodiments, the antibody or antibody fragment specifically binds to a fibroblast growth factor (FGF), e.g., a human FGF. In some embodiments, the antibody or antibody fragment specifically binds to one or more immunodominant epitope(s) within a FGF protein.

In some embodiments, the antibody or antibody fragment specifically binds to a fibroblast growth factor receptor (FGFR), e.g., a human FGFR. In some embodiments, the antibody or antibody fragment specifically binds fibroblast growth factor receptor 2 (FGFR2) protein, e.g., a human FGFR2 protein, for example, a FGFR2b protein. In some embodiments, the antibody or antibody fragment specifically binds a fibroblast growth factor receptor 3 (FGFR3) protein, e.g., a human FGFR3 protein. In some embodiments, the antibody or antibody fragment specifically binds to one or more immunodominant epitope(s) within a FGFR protein, a FGFR2 protein or a FGFR3 protein.

In some embodiments, the antibody specifically binds to a receptor tyrosine kinase cMET protein. In some embodiments, the antibody specifically binds to one or more immunodominant epitope(s) within a receptor tyrosine kinase cMET protein.

In some embodiments, the antibody specifically binds to a CD47 protein, e.g., a human CD47 protein. In some embodiments, the antibody specifically binds to one or more immunodominant epitope(s) within a CD47 protein.

In some embodiments, the antibody specifically binds to an immune checkpoint inhibitor. In some embodiments, the antibody binds to one or more immunodominant epitope(s) within an immune checkpoint inhibitor. In some embodiments, the antibody specifically binds to a programmed death protein, e.g., a human PD-1. In some embodiments, the antibody specifically binds to one or more immunodominant epitope(s) within PD-1 protein.

In some embodiments, the antibody specifically binds to a programmed death ligand-1 (PD-L1) protein, e.g., a human PD-L1. In some embodiments, the antibody specifically binds to one or more immunodominant epitope(s) within PD-L1 protein.

In some embodiments, the antibody binds to TIM3. In some embodiments, the antibody binds to one or more immunodominant epitope(s) within TIM3.

In some embodiments, the antibody specifically binds to a lectin. In some embodiments, the antibody specifically binds to one or more immunodominant epitope(s) within a lectin. In some embodiments, the antibody binds to SIGLEC. In some embodiments, the antibody binds to one or more immunodominant epitope(s) within SIGLEC. In some embodiments, the antibody binds to a cytokine receptor. In some embodiments, the antibody binds to a one or more immunodominant epitope(s) within cytokine receptor. In some embodiments, the antibody binds to sIL6R. In some embodiments, the antibody binds to one or more immunodominant epitope(s) within sIL6R. In some embodiments, the antibody binds to a cytokine. In some embodiments, the antibody binds to one or more immunodominant epitope(s) within a cytokine. In some embodiments, the antibody binds to MCP-1, TNF (e.g., a TNF-alpha), IL1a, IL1b, IL4, IL5, IL6, IL12/IL23, IL13, IL17 or p40. In some embodiments, the antibody binds to one or more immunodominant epitope(s) within MCP-1, TNF (e.g., a TNF-alpha), IL1a, IL1b, IL4, IL5, IL6, IL12/IL23, IL13, IL17 or p40.

In some embodiments, the antibody binds to a major histocompatibility protein (e.g., a MHC class I or class II molecule). In some embodiments, the antibody binds to one or more immunodominant epitope(s) within a major histocompatibility protein (e.g., a MHC class I or class II molecule). In some embodiments, the antibody binds to beta 2 microglobulin. In some embodiments, the antibody binds to one or more immunodominant epitope(s) within beta 2 microglobulin.

### 6.1.3.2 Small Molecules

In some embodiments, the target-binding moiety of the bifunctional compound of this disclosure is a small molecule that specifically binds to a target molecule, such as a target protein. In some embodiments, the bifunctional compound includes a small molecule inhibitor or ligand of a target protein. A small molecule target-binding moiety can be covalently linked to one or more IGF-2 polypeptides via a linker. The linker can be attached to the small molecule via substitution at any suitable site of the small molecule such that binding to the target protein is substantially retained.

In some embodiments, the target-binding moiety is a small molecule inhibitor or antagonist of a target protein (e.g., as described herein).

In some embodiments, the target-binding moiety is a small molecule inhibitor or antagonist of VEGF.

In some embodiments, the target-binding moiety is a small molecule inhibitor or antagonist of TNF protein (e.g., TNF-alpha).

In some embodiments, the target-binding moiety is a small molecule inhibitor or antagonist of PD-L1.

In some embodiments, the target-binding moiety is a small molecule inhibitor or antagonist of EGFR protein, a VEGFR protein, a FGFR2 protein or a FGFR3 protein.

### 6.1.1. Bifunctional IGF2-Target Binding Moiety Conjugates

In some embodiments, the bifunctional compound of this disclosure is a conjugate of an IGF-2 polypeptide and a target-binding moiety, and is of formula (I):

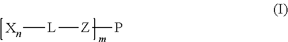

or a pharmaceutically acceptable salt thereof,
wherein:
X is an IGF-2 polypeptide (e.g., as described herein);
n is 1 to 100;
L is an optional linker;
Z is a residual linking moiety resulting from the attachment of Xn (or L, if present) to P via a chemoselective ligation group;
P is the target-binding moiety; and
m is 1 to 20.

In some embodiments of formula (I), n is 1 to 20. In some embodiments of formula (I), n is 1 to 6. In some embodiments of formula (I), n is 1 to 5. In some embodiments of formula (I), n is 1 to 4. In some embodiments of formula (I), n is 1 to 3. In some embodiments of formula (I), n is 2. In some embodiments of formula (I), n is 1.

In some embodiments of formula (I), the linker (L) is present and is a linear bivalent linker (i.e., n is 1). In some embodiments of formula (I), the linker (L) is trivalent and n is 2. In some embodiments of formula (I), the linker (L) is multivalent (i.e., n is >1). A multivalent linker can also be referred to as a branched linker.

In some embodiments of formula (I), n is 1 and m is 1 such that the bifunctional compound comprises a ratio of IGF-2 polypeptide to target-binding moiety is 1:1.

In some embodiments of formula (I), n is 1 and m is >1, such that the bifunctional compound includes two or more IGF-2 polypeptides linked to one target-binding moiety via linear linkers.

In some embodiments of formula (I), n is >1 and m is 1, such that the bifunctional compound includes a branched linker connecting two or more IGF-2 polypeptides to one target-binding moiety.

In some embodiments of formula (I), the target-binding moiety (P) is an antibody or antibody fragment. IGF-2 polypeptide can be site-specifically covalently linked to the antibody or antibody fragment, e.g., via the residual moiety Z.

In some embodiments of formula (I), the IGF-2 polypeptide is covalently linked to the antibody or antibody fragment via a site-specific cysteine modification on the antibody or antibody fragment and a thiol-reactive chemoselective ligation group. In some embodiments of formula (I), P is an antibody that includes a L443C modification, where the cysteine residue is covalently linked to an IGF-2 polypeptide via a residual linking moiety Z that results from conjugation of a thiol-reactive chemoselective ligation group with the cysteine sidechain. In some embodiments, thiol-reactive chemoselective ligation group is maleimide, and Z is a thiosuccinimide.

In some embodiments of formula (I), the IGF-2 polypeptide is covalently linked to the antibody or antibody fragment via one or more lysine residues of the antibody or antibody fragment and an amine-reactive chemoselective ligation group. In some embodiments, amine-reactive chemoselective ligation group is an active ester, such as a fluorophenyl ester (e.g., PFP ester) or a NHS ester. In such cases, the residual moiety Z of formula (I) can be an amide attached to the sidechain of a lysine residue of the antibody or antibody fragment.

The number of “X” moieties per each unit of “Xn-L-” or “Xn-” is represented by “n” in formulas (I). The term “valency” or “valencies” refers to the number of “X” moieties per unit (“n”). It is understood that loading is not necessarily equivalent to the number of “X” moieties per conjugate molecule. By means of example, where there is one “X” moiety per unit (n=1; valency is “1”), and one “Xn-L-” unit per conjugate (m=1), there will be 1×1=1 “X” moiety per conjugate. However, where there are two “X” moieties per unit (n=2; valency is “2”), and four “Xn-L-” units per conjugate (m=4), there will be 2×4=8 “X” moieties per conjugate. Accordingly, for formula (I) described herein, the total number of “X” moieties per bifunctional compound will be n×m. The term “total valency” or “total valencies” refers to the total number of “X” moieties per conjugate molecule (n×m; total valency).

In some embodiments of formula (I), m is 1 to 20. In some embodiments of formula (I), m is 1 to 10. In some embodiments of formula (I), m is 2 to 10. In some embodiments of formula (I), m is 2 to 6.

In some embodiments of formula (I), m represents an average loading of the linked IGF-2 polypeptide (Xn-L-) on the target-binding moiety P. In some embodiments of formula (I), the bifunctional compound comprises a ratio of IGF-2 polypeptide to target-binding moiety of about 2:1 to about 10:1. In some embodiments of formula (I), the bifunctional compound comprises a ratio of IGF-2 polypeptide to target-binding moiety is about 2:1.

### 6.1.1.1 Linking Moieties

The IGF-2 polypeptide can be covalently linked to the target-binding moiety via a linking moiety.

The terms “linker”, “linking moiety” and “linking group” are used interchangeably and refer to a linking moiety that covalently connects two or more moieties or compounds, such as an IGF-2 polypeptide and the target-binding moiety of interest. In some cases, the linker is divalent and connects two moieties. In certain cases, the linker is a branched linking group that is trivalent or of a higher multivalency. In some cases, the linker that connects the two or more moieties has a linear or branched backbone of 500 atoms or less (such as 400 atoms or less, 300 atoms or less, 200 atoms or less, 100 atoms or less, 50 atoms or less, or even 20 atoms or less) in length, e.g., as measured between the two or more moieties. A linking moiety may be a covalent bond that connects two groups or a linear or branched chain of between 1 and 500 atoms in length, for example of about 1, 2, 3, 4, 5, 6, 8, 10, 12, 14, 16, 18, 20, 30, 40, 50, 100, 150, 200, 300, 400 or 500 carbon atoms in length, where the linker may be linear, branched, cyclic or a single atom. In certain cases, one, two, three, four, five or more, ten or more, or even more carbon atoms of a linker backbone may be optionally substituted with heteroatoms, e.g., sulfur, nitrogen or oxygen heteroatom.

The bonds between backbone atoms may be saturated or unsaturated, usually not more than one, two, or three unsaturated bonds will be present in a linker backbone. The linker may include one or more substituent groups, for example an alkyl, aryl or alkenyl group. In certain instances, when the linker includes a polyethylene glycol (PEG) group, every third atom of that segment of the linker backbone is substituted with an oxygen. A linker may include, without limitations, one or more of the following: oligo(ethylene glycol), ether, thioether, disulfide, amide, carbonate, carbamate, tertiary amine, alkyl which may be straight or branched, e.g., methyl, ethyl, n-propyl, 1-methylethyl (iso-propyl), n-butyl, n-pentyl, and the like. The linker backbone may include a cyclic group, for example, an aryl, a heterocycle, a cycloalkyl group or a heterocycle group, where 2 or more atoms, e.g., 2, 3 or 4 atoms, of the cyclic group are included in the backbone.

In some embodiments, the linker includes one or more linking moieties independently selected from —C1-6-alkylene-, —NHCO—C1-6-alkylene-, —CONH—C1-6-alkylene-, —O(CH2)p-, —(OCH2CH2)p-, —NHCO—, —CONH—, —NHSO2-, —SO2NH—, —CO—, —SO2-, —O—, —S—, pyrrolidine-2,5-dione, —NH—, and —NMe-, wherein p is 1 to 10.

In some embodiments, a “linker” or linking moiety is derived from a molecule with two reactive termini, one for conjugation to a target-binding moiety of interest, e.g., a biomolecule (e.g., an antibody), and the other for conjugation to the IGF-2 polypeptide. It is understood that the linker may encompass one or more linking functional groups that are the residual product of a coupling reaction between two chemoselective ligation groups (e.g., as described herein). Residual linking functional groups derived from any of the conjugation chemistries described herein can be incorporated into the linkers of the compounds and conjugates of this disclosure.

### 6.1.1.2 Chemoselective Ligation Groups

A variety of chemoselective ligation groups, can be utilized to connect the IGF-2 polypeptide and target-binding moiety via an optional linker, and thus can be incorporated into bifunctional compounds of this disclosure, as part of residual linking functional groups. A chemoselective ligation group is a group having a reactive functionality or functional group capable of conjugation to a compatible group of a second moiety. For example, chemoselective ligation groups (or precursors thereof) may be one of a pair of groups associated with a conjugation chemistry such as azido-alkyne click chemistry, copper free click chemistry, Staudinger ligation, tetrazine ligation, hydrazine-iso-Pictet-Spengler (HIPS) ligation, cysteine-reactive ligation chemistry (e.g., thiol-maleimide, thiol-haloacetamide or alkyne hydrothiolation), amine-active ester amido bond coupling, reductive amination, dialkyl squarate chemistry, etc.

Chemoselective ligation groups that may be utilized in linking two moieties, include, but are not limited to, amine (e.g., a N-terminal amine or a lysine sidechain amine group of a IGF-2 polypeptide), azide, aryl azide, alkynyl (e.g., ethynyl or cyclooctyne or derivative), active ester (e.g., N-hydroxysuccinimide (NHS) ester, sulfo-NHS ester or pentafluorophenyl (PFP) ester or thioester), haloacetamide (e.g., chloroacetamide, iodoacetamide or bromoacetamide), chloroacetyl, bromoacetyl, hydrazide, maleimide, vinyl sulfone, 2-sulfonyl pyridine, cyano-alkyne, thiol (e.g., a cysteine residue), disulfide or protected thiol, isocyanate, isothiocyanate, aldehyde, ketone, aminooxy or alkoxyamine, hydrazide, hydroxylamino, phosphine, HIPS hydrazinyl-indolyl group, or aza-HIPS hydrazinyl-pyrrolo-pyridinyl group, tetrazine, cyclooctene, squarate, and the like.

A chemoselective ligation group is generally capable of spontaneous conjugation with a compatible chemical group when the two groups come into contact under suitable conditions (e.g., copper free Click chemistry conditions). In some embodiments, the chemoselective ligation group is capable of conjugation to a compatible chemical group when the two groups come into contact in the presence of a catalyst or other reagent (e.g., copper catalyzed Click chemistry conditions).

In some embodiments, the chemoselective ligation group is a photoactive ligation group. For example, upon irradiation with ultraviolet light, a diazirine group can form reactive carbenes, which can insert into C—H, N—H, and O—H bonds of a second moiety.

In some embodiments, the chemoselective ligation group is capable forming a covalent bond to a polypeptide (e.g., with an amino acid sidechain of a polypeptide having a compatible reactive group). In some embodiments, an chemoselective ligation group is an amino-reactive chemoselective ligation group (e.g., an active ester) that provides for conjugation to an amine group of a target-binding moiety. In some embodiments, a chemoselective ligation group is a thiol-reactive chemoselective ligation group (e.g., maleimide or haloacetamide) that provides for conjugation to a thiol group of a target-binding moiety. Thus, the bifunctional compound can include a residual moiety that results from the covalent linkage of a thiol with a thiol-reactive chemoselective ligation group, or from an amine and an amine-reactive chemoselective ligation group (an amide linkage).

### 6.1.2. Fusion Proteins

In some embodiments, the bifunctional compound of this disclosure is a chimeric fusion of the IGF-2 polypeptide and a polypeptide moiety that specifically binds the target moiety. In some embodiments, the polypeptide target-binding moiety is selected from target-binding peptide, target-binding protein (e.g., engineered protein domain or scaffolded protein binder), or target-binding antibody or antibody fragment.

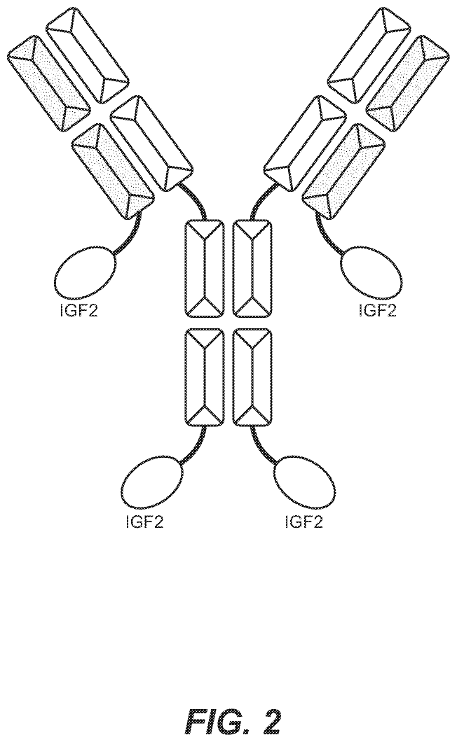

FIG. 2 illustrates an exemplary bifunctional compound that is a fusion protein where the IGF-2 polypeptides are incorporated into the architecture of a target-binding antibody via fusion to the C-terminals of each of the immunoglobulin heavy and light chains of the antibody. Fusion of the antibody or antibody fragment and an IGF-2 polypeptide can be achieved via an optional spacer sequence.

In some embodiments, the bifunctional compound is a fusion protein that includes a spacer or intervening sequence of amino acid resides between the IGF-2 polypeptide and the proteinaceous target-binding moiety. The term “spacer” (also referred to as “linker”) refers to an intervening peptide sequence between two protein moieties in a fusion protein. A spacer is generally designed to be flexible or to interpose a structure, such as an alpha-helix, between the two protein moieties. A spacer can be relatively short, such for example, the sequence Gly-Ala-Pro (GAP) (SEQ ID NO: 14), Gly-Gly-Gly-Gly-Ser (GGGGS) (SEQ ID NO: 15), Gly-Gly-Gly-Gly-Ala (GGGGA) (SEQ ID NO: 16), or Gly-Gly-Gly-Gly-Gly-Pro (GGGGGP) (SEQ ID NO: 17), or the reverse of such spacer sequences depending on where the spacer is located. Alternatively, a spacer can be longer, such as, for example, 10-25 amino acids in length, 25-50 amino acids in length, or 35-55 amino acids in length.

In some embodiments, the bifunctional compound incudes a spacer peptide located between the polypeptide target-binding moiety and the IGF-2 polypeptide, wherein the spacer peptide includes an amino acid sequence selected from:

(SEQ ID NO: )
GGGGSGGGGSGGGGSGGGGSGGGPS,
(SEQ ID NO: )
GAPGGSPAGSPTSTEEGTSESATPESGPGTSTEPSEGSAPGSPAGSPTS
TGPSGAP,
(SEQ ID NO: )
GAPGGGSPAPAPTPAPAPTPAPAGGGPSGAP,
(SEQ ID NO: )
GAPGGGSPAEAAAKEAAAKEAAAKEAAAKEAAAKAPSGGGGAP,
and
(SEQ ID NO: )
GGGGAGGGGAGGGGAGGGGAGGGPS.

In some embodiments, the bifunctional compound is a fusion protein that includes an antibody as the target-binding moiety. In some embodiments, the bifunctional compound is a fusion protein that includes an antibody fragment as the target-binding moiety. The IGF-2 polypeptide can be fused to the antibody or antibody fragment at any convenient location(s).

In some embodiments, the IGF-2 polypeptide is fused to the antibody or antibody fragment via the IGF-2 polypeptide's C-terminal amino acid residue, and an optional spacer. In some embodiments, the IGF-2 polypeptide is fused to the antibody or antibody fragment via the IGF-2 polypeptide's N-terminal amino acid residue, and an optional spacer. In some embodiments, the IGF-2 polypeptide is inserted into the antibody or antibody fragment sequence at a site, and thus is fused via both its N-terminal and C-terminal residues, via optional spacers.

This disclosure also provides nucleic acids encoding the IGF-2 polypeptide containing fusion protein as described in various embodiments here. This disclosure further provides various cells containing the nucleic acid of the disclosure.

In another aspect, the present disclosure provides a method of producing a bifunctional compound fusion protein including a step of culturing mammalian cells in a cell culture medium, wherein the mammalian cells carry the nucleic acid of this disclosure that encodes the bifunctional compound, (e.g., as described herein), and the culturing is performed under conditions that permit expression of the bifunctional compound fusion protein. Any mammalian cell or cell type susceptible to cell culture, and to expression of polypeptides, may be utilized in accordance with the present disclosure. The bifunctional compound fusion protein of this disclosure can also be expressed in a variety of non-mammalian host cells.

### 6.2. Methods

Aspects of this disclosure include methods for internalizing an extracellular target in a cell comprising a M6PR cell surface receptor, e.g., CI-M6PR. The method can include contacting a cellular sample or biological system comprising the cell and an extracellular target with an effective amount of a bifunctional compound (e.g., as described herein) that specifically binds the target and specifically binds the cell surface receptor to facilitate cellular uptake of the target moiety.

In some embodiments of the method, the bifunctional compound described herein removes a target protein from a cell's surface. In some embodiments of the method, the bifunctional compound described herein removes a target protein from the extracellular milieu. For example, the methods can provide for removal of the target protein from the surface of a cell, or from the extracellular space, by sequestering the target protein in the cell's lysosome. In some embodiments, the method leads to sequestering the target protein in the cell's lysosome and degrading the target protein.

Removal of a target protein may refer to reduction, or depletion, of the target protein from the cell surface or from the extracellular space, or the extracellular milieu. In some embodiments, the method is a method of reducing the amount or level of a target protein in a biological system or cellular sample.

In one aspect, provided herein are methods of using the bifunctional compounds described herein to degrade a target protein of interest, e.g., through a cell's lysosomal pathway.

In another aspect, provided herein are methods of depleting or reducing levels of a target protein by administering to a subject in need thereof an effective amount of a bifunctional compound or pharmaceutically acceptable salt described herein, or a pharmaceutical composition described herein. In certain embodiments, the subject is a mammal (e.g., human).

### 6.2.1. Cells and Cell-Surface Receptors

In some embodiments, the bifunctional compound and bound target moiety can be taken up by a variety of cell types having M6PR cell surface receptor and transported to the lysosome.

In some embodiments, the bifunctional compound is used to target particular tissues having cell that express high levels of a M6PR cell surface receptor in a biological system. Thus, the bifunctional compounds can exhibit cell tropism, or tissue tropism.

In some embodiments, the target cell is a mammalian cell. In some embodiments, the cell is a muscle cell, neural cell, liver cell, cardiac cell, lung cell, immune cell, or kidney cell.

### 6.2.2. Targets

Aspects of this disclosure include methods for reducing levels of an extracellular target moiety in a cell. In some embodiments, the target moiety is a membrane bound target protein. In some embodiments, the target moiety is a soluble target protein. The bifunctional compound of this disclosure specifically binds the target protein and facilitates its internalization and degradation in cells of the subject

In some embodiments, the target moiety is an extracellular target protein associated with a disease or disorder. Aspects of this disclosure thus include methods of treating a disease or disorder associated with a target protein. In certain embodiments, the disease or disorder is treated by depletion of the target protein by degradation through the lysosomal pathway.

In some embodiments, the disease or disorder is treated by depletion of certain proteins, for example, soluble proteins, e.g., secreted proteins, cell surface proteins (for example, cell surface receptor proteins, e.g., tyrosine kinase receptors, soluble cytokine receptors, and immune checkpoint receptors, e.g., EGFR, VEGFR, FGFR, and PD-L1), lectins, complements, lipoproteins, transport proteins, MHC class I and class II molecules, cytokines, chemokines, and/or receptors, or fragments or subunits of any of the foregoing.

In some embodiments, the methods of treating a disease or disorder including administering to a subject, e.g., a human, in need thereof an effective amount of a bifunctional compound or pharmaceutically acceptable salt described herein, or a pharmaceutical composition described herein. The terms “subject” and “patient” are used interchangeably. A subject can be a mammal such as a non-primate (e.g., cows, pigs, horses, cats, dogs, goats, rabbits, rats, mice, etc.) or a primate (e.g., monkey and human), for example a human. In certain embodiments, the subject is a mammal, e.g., a human, diagnosed with a disease or disorder provided herein. In another embodiment, the subject is a mammal, e.g., a human, at risk of developing a disease or disorder provided herein. In a specific embodiment, the subject is human.

In some embodiments, the disease or disorder is an inflammatory or autoimmune disease. In certain embodiments, the disease or disorder is an inflammatory disease. In certain embodiments, the disease or disorder is an autoimmune disease.

In some embodiments, the disease or disorder is a cancer. In some embodiments, when the disorder or disease is cancer, “effective amount” or “therapeutically effective amount” mean that amount of a compound or conjugate or pharmaceutical composition provided herein which, when administered to a human suffering from a cancer, is sufficient to effect treatment for the cancer. Treating” or “treatment” of the cancer includes one or more of:
(1) limiting/inhibiting growth of the cancer, e.g. limiting its development;
(2) reducing/preventing spread of the cancer, e.g. reducing/preventing metastases;
(3) relieving the cancer, e.g. causing regression of the cancer,
(4) reducing/preventing recurrence of the cancer; and
(5) palliating symptoms of the cancer.

In some embodiments, the cancer is selected from the group consisting of bladder cancer, breast cancer, cervical cancer, cholangiocarcinoma, endometrial cancer, hepatocellular carcinoma, kidney cancer, melanoma, myeloid neoplasms, non-small cell lung cancer (NSCLC), Ewing's sarcoma, and Hodgkin's Lymphoma. In some embodiments, the cancer is a solid tumor.

### 6.3. Definitions

The terms “protein” and “polypeptide” are used interchangeably. Proteins may include moieties other than amino acids (e.g., may be glycoproteins, etc.) and/or may be otherwise processed or modified. Those of ordinary skill in the art will appreciate that a “protein” can be a complete protein chain as produced by a cell (with or without a signal sequence), or can be a protein portion thereof. Those of ordinary skill will appreciate that a protein can sometimes include more than one protein chain, for example non-covalently or covalently attached, e.g., linked by one or more disulfide bonds or associated by other means. Polypeptides may contain 1-amino acids, d-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. In some embodiments, proteins are antibodies, antibody fragments, biologically active portions thereof, and/or characteristic portions thereof.

The terms “antibody” and “immunoglobulin” are terms of art and can be used interchangeably herein, and refer to a molecule with an antigen binding site that specifically binds an antigen. In some embodiments, an isolated antibody (e.g., monoclonal antibody) described herein, or an antigen-binding fragment thereof, which specifically binds to a protein of interest is conjugated to one or more IGF-2 polypeptides, for example, via a linker.

An “antigen” is a moiety or molecule that contains an epitope to which an antibody can specifically bind. Thus, an antigen is also is specifically bound by an antibody. In a specific embodiment, the antigen, to which an antibody described herein binds, is a target protein of interest, for example, EGFR (e.g., human EGFR), or a fragment thereof, or for example, an extracellular domain of EGFR (e.g., human EGFR).

An “epitope” is a term known in the art and refers to a localized region of an antigen to which an antibody can specifically bind. An epitope can be a linear epitope of contiguous amino acids or can comprise amino acids from two or more non-contiguous regions of the antigen.

The terms “binds,” “binds to,” “specifically binds” or “specifically binds to” in the context of antibody binding refer to antibody binding to an antigen (e.g., epitope) as such binding is understood by one skilled in the art. For example, a molecule that specifically binds to an antigen may bind to other polypeptides, generally with lower affinity as determined by, e.g., immunoassays, Biacore™, KinExA 3000 instrument (Sapidyne Instruments, Boise, ID), or other assays known in the art. In some embodiments, molecules that specifically bind to an antigen bind to the antigen with an affinity (Kd) that is at least 2 logs, 2.5 logs, 3 logs, 4 logs lower (higher affinity) than the Kd when the molecules bind to another antigen. In some embodiments, molecules that specifically bind to an antigen do not cross react with other proteins. In some embodiments, where EGFR is the protein of interest, molecules that specifically bind to an antigen do not cross react with other non-EGFR proteins.

Antibodies can include, for example, monoclonal antibodies, recombinantly produced antibodies, monospecific antibodies, multispecific antibodies (including bispecific antibodies), human antibodies, humanized antibodies, chimeric antibodies, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain/antibody heavy chain pair, an antibody with two light chain/heavy chain pairs (e.g., identical pairs), intrabodies, heteroconjugate antibodies, single domain antibodies, monovalent antibodies, bivalent antibodies (including monospecific or bispecific bivalent antibodies), single chain antibodies, or single-chain Fvs (scFv), camelized antibodies, affybodies, Fab fragments, F(ab′) fragments, F(ab′)2 fragments, disulfide-linked Fvs (sdFv), anti-idiotypic (anti-Id) antibodies (including, e.g., anti-anti-Id antibodies), and epitope-binding fragments of any of the above.

Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In some embodiments, antibodies described herein are IgG antibodies (e.g., human IgG), or a class (e.g., human IgG1, IgG2, IgG3 or IgG4) or subclass thereof.

In a particular embodiment, an antibody is a 4-chain antibody unit comprising two heavy (H) chain/light (L) chain pairs, wherein the amino acid sequences of the H chains are identical and the amino acid sequences of the L chains are identical. In a specific embodiment, the H and L chains comprise constant regions, for example, human constant regions. In a yet more specific embodiment, the L chain constant region of such antibodies is a kappa or lambda light chain constant region, for example, a human kappa or lambda light chain constant region. In another specific embodiment, the H chain constant region of such antibodies comprise a gamma heavy chain constant region, for example, a human gamma heavy chain constant region. In a particular embodiment, such antibodies comprise IgG constant regions, for example, human IgG constant regions.

The term “constant region” or “constant domain” is a well-known antibody term of art (sometimes referred to as “Fc”), and refers to an antibody portion, e.g., a carboxyl terminal portion of a light and/or heavy chain which is not directly involved in binding of an antibody to antigen but which can exhibit various effector functions, such as interaction with the Fc receptor. The terms refer to a portion of an immunoglobulin molecule having a generally more conserved amino acid sequence relative to an immunoglobulin variable domain.

The term “heavy chain” when used in reference to an antibody can refer to any distinct types, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

The term “light chain” when used in reference to an antibody can refer to any distinct types, e.g., kappa (κ) of lambda (λ) based on the amino acid sequence of the constant domains. Light chain amino acid sequences are well known in the art. In specific embodiments, the light chain is a human light chain.

The term “monoclonal antibody” is a well-known term of art that refers to an antibody obtained from a population of homogenous or substantially homogeneous antibodies. The term “monoclonal” is not limited to any particular method for making the antibody. Generally, a population of monoclonal antibodies can be generated by cells, a population of cells, or a cell line. In specific embodiments, a “monoclonal antibody,” as used herein, is an antibody produced by a single cell (e.g., hybridoma or host cell producing a recombinant antibody), wherein the antibody specifically binds to an epitope as determined, e.g., by ELISA or other antigen-binding or competitive binding assay known in the art or in the Examples provided herein. In particular embodiments, a monoclonal antibody can be a chimeric antibody or a humanized antibody. In certain embodiments, a monoclonal antibody is a monovalent antibody or multivalent (e.g., bivalent) antibody. In particular embodiments, a monoclonal antibody is a monospecific or multispecific antibody (e.g., bispecific antibody).

The terms “variable region” or “variable domain” refer to a portion of an antibody, generally, a portion of a light or heavy chain, typically about the amino-terminal 110 to 120 amino acids in the mature heavy chain and about 90 to 100 amino acids in the mature light chain. Variable regions comprise complementarity determining regions (CDRs) flanked by framework regions (FRs). Generally, the spatial orientation of CDRs and FRs are as follows, in an N-terminal to C-terminal direction: FR1-CDR1-FR2-CDR2-FR3-CDR3-FR4. Without wishing to be bound by any particular mechanism or theory, it is believed that the CDRs of the light and heavy chains are primarily responsible for the interaction of the antibody with antigen and for the specificity of the antibody for an epitope. In a specific embodiment, numbering of amino acid positions of antibodies described herein is according to the EU Index, as in Kabat et al. (1991) Sequences of Proteins of Immunological Interest, Fifth Edition, U.S. Department of Health and Human Services, NIH Publication No. 91-3242. In certain embodiments, the variable region is a human variable region.

In certain aspects, the CDRs of an antibody can be determined according to (i) the Kabat numbering system (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196: 901-917; Al-Lazikani et al., 1997, J. Mol. Biol., 273: 927-948; Chothia et al., 1992, J. Mol. Biol., 227: 799-817; Tramontano et al., 1990, J. Mol. Biol. 215(1):175-82; U.S. Pat. No. 7,709,226; and Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001)); or (iii) the ImMunoGeneTics (IMGT) numbering system, for example, as described in Lefranc, 1999, The Immunologist, 7: 132-136 and Lefranc et al., 1999, Nucleic Acids Res., 27: 209-212 (“IMGT CDRs”); or (iv) the AbM numbering system, which will be referred to herein as the “AbM CDRs”, for example as described in MacCallum et al., 1996, J. Mol. Biol., 262: 732-745. See also, e.g., Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001); or (v) the Contact numbering system, which will be referred to herein as the “Contact CDRs” (the Contact definition is based on analysis of the available complex crystal structures (bioinf.org.uk/abs) (see, e.g., MacCallum et al., 1996, J. Mol. Biol., 262:732-745)).

The terms “full length antibody,” “intact antibody” and “whole antibody” are used herein interchangeably to refer to an antibody in its substantially intact form, and are not antibody fragments as defined below. The terms particularly refer to an antibody with heavy chains that contain the Fc region.

“Antibody fragments” include only a portion of an intact antibody, wherein the portion retains at least one, two, three and as many as most or all of the functions normally associated with that portion when present in an intact antibody. In one aspect, an antibody fragment comprises an antigen binding site of the intact antibody and thus retains the ability to bind antigen. In another aspect, an antibody fragment, such as an antibody fragment that comprises the Fc region, retains at least one of the biological functions normally associated with the Fc region when present in an intact antibody. Such functions may include FcRn binding, antibody half life modulation, conjugate function and complement binding. In another aspect, an antibody fragment is a monovalent antibody that has an in vivo half life substantially similar to an intact antibody. For example, such an antibody fragment may comprise on antigen binding arm linked to an Fc sequence capable of conferring in vivo stability to the fragment.

The terms “administer”, “administration”, or “administering” refer to the act of injecting or otherwise physically delivering a substance (e.g., a compound or pharmaceutical composition provided herein) to a subject or a patient (e.g., human), such as by mucosal, topical, intradermal, parenteral, intravenous, intramuscular delivery and/or any other method of physical delivery described herein or known in the art. In a particular embodiment, administration is by intravenous infusion.

The terms “effective amount” or “therapeutically effective amount” refer to an amount of a therapeutic (e.g., a conjugate or pharmaceutical composition provided herein) which is sufficient to treat, diagnose, prevent, delay the onset of, reduce and/or ameliorate the severity and/or duration of a given condition, disorder or disease and/or a symptom related thereto. These terms also encompass an amount necessary for the reduction, slowing, or amelioration of the advancement or progression of a given disease, reduction, slowing, or amelioration of the recurrence, development or onset of a given disease, and/or to improve or enhance the prophylactic or therapeutic effect(s) of another therapy or to serve as a bridge to another therapy. In some embodiments, “effective amount” as used herein also refers to the amount of a conjugate described herein to achieve a specified result.

M6P refers to mannose-6-phosphate. M6PR refers to mannose-6-phosphate receptor. CI-M6PR refers to cation independent mannose-6-phosphate receptor.

IGF1R refers to insulin-like growth factor 1 receptor.

The terms “treatment” and “therapy” can refer to any protocol(s), method(s), compositions, formulations, and/or agent(s) that can be used in the prevention, treatment, management, or amelioration of a disease or disorder or symptom thereof (e.g., a disease or disorder provided herein or one or more symptoms or condition associated therewith). In certain embodiments, the terms “therapies” and “therapy” refer to drug therapy, adjuvant therapy, radiation, surgery, biological therapy, supportive therapy, and/or other therapies useful in treatment, management, prevention, or amelioration of a disease or disorder or one or more symptoms thereof. In certain embodiments, the term “therapy” refers to a therapy other than a conjugate described herein or pharmaceutical composition thereof.

A “variant” is a polypeptide having one or more different amino acid residues as compared to a corresponding parental polypeptide sequence, or a fragment thereof having a similar or identical length to the variant. In some embodiments, a parental polypeptide sequence is the wild type or naturally occurring polypeptide sequence. In some embodiments, a variant polypeptide has at least 70% sequence identity to its parent sequence, or a fragment thereof, such as at least 75%, at least 80%, at least 85%, at least 90%, or at least 95% sequence identity.

Unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 10%, 9%, 8%, 7%, 6%, 5%, 4%, 3%, 2%, 1%, 0.5%, 0.4%, 0.3%, 0.25%, 0.2%, 0.1% or 0.05% of a given value or range. In certain embodiments, where an integer is required, the term “about” means within plus or minus 10% of a given value or range, rounded either up or down to the nearest integer.

### 7. EXAMPLES

### 7.1. Example 1: Preparation of Exemplary Bifunctional Compounds

### 7.1.1. Preparation of Omalizumab-IGF2 Bifunctional Compound.

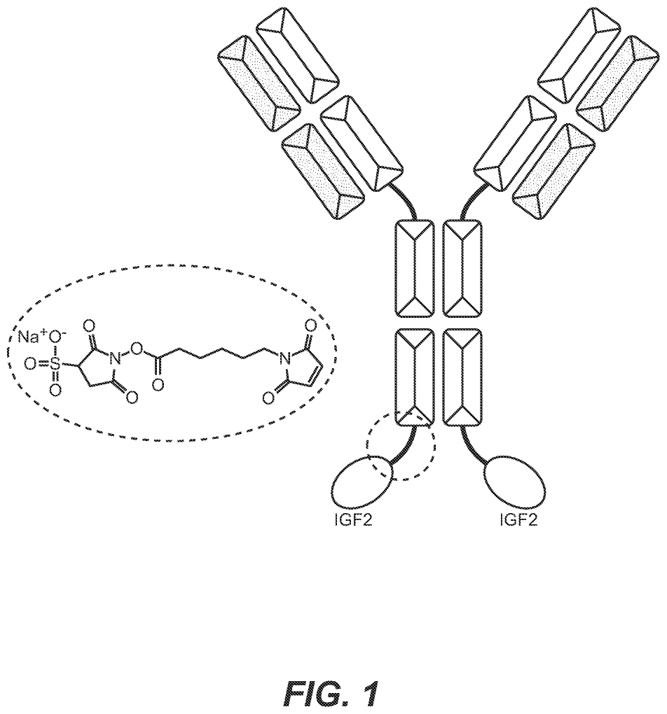

A bifunctional compound (FIG. 1) was prepared via conjugation of omalizumab (anti-IgE antibody) and IGF-2 polypeptide using the bivalent linker 6-maleimidocaproic acid sulfo-NHS. Recombinant IGF-2 polypeptide was obtained from R&D Systems, and conjugated with the NHS ester of the bivalent linker, e.g., at the N-terminal amine group and/or the sidechain amine group of the lysine residue of IGF-2. The linker modified IGF-2 polypeptide was then conjugated with the antibody. Omalizumab antibody having site-specific mutation L443C was used for conjugation of the cysteine sidechain group to the maleimide group of the linker.

Excess linker is removed by size exclusion chromatography (Superdex 200 Increase, Cat #28990944). The purity of the conjugate is determined through size exclusion high performance liquid chromatography (SEC-HPLC). The ratio of IGF-2 polypeptide to antibody in the conjugates is determined using mass spectrometry LC/MS (Sciex 5600+, Acquity UPLC BEH C4 column, Cat #186004495).

For comparison, conjugates of the omalizumab antibody with alternative cell surface receptor ligands were prepared using similar methods, including a mannose-6-phosphate-ligand (M6P)-linker precursor or a M6Pn ligand-linker precursor (see e.g., International application No. PCT/US2021/012846).

### 7.1.1. Preparation of Fluorescently Labelled Compounds

The omalizumab antibody conjugates were labelled with Alexa Fluor 488 (AF488) Protein Labeling Kit (Invitrogen) per the manufacturer's protocol. In brief, antibodies to be labeled were diluted to 2 mg/mL in PBS to a total volume of 500 μL. A 15 DOL (degree of labeling) was used for the conjugation with the fluorophore. Free dye was removed by pre-wetting an Amicon 30 kDa filter with PBS. After incubation, the conjugation reaction mixture was added to the filter and spun at high speed for 10 minutes. Retained solution was resuspended in PBS to a final volume of 1 mL and stored at 4° C.

### 7.2. Example 2: Assessment of Exemplary Bifunctional Compounds

### 7.2.1. In Vitro Cell Uptake Assay

Omalizumab was conjugated to M6P, M6Pn, or IGF-2 polypeptide as described above or left unconjugated (UNLB). The omalizumab compositions were then fluorescently labelled with Alexa 488 fluorescent dye as described above.

Uptake of the omalizumab-AF488 compositions was evaluated in Jurkat (human), C2C12 (mouse) and primary mouse fibroblasts cells. Cells were incubated for 1 hour with the compositions and then cellular uptake was assessed by measuring mean fluorescent intensity (MFI) of cells using flow cytometry.

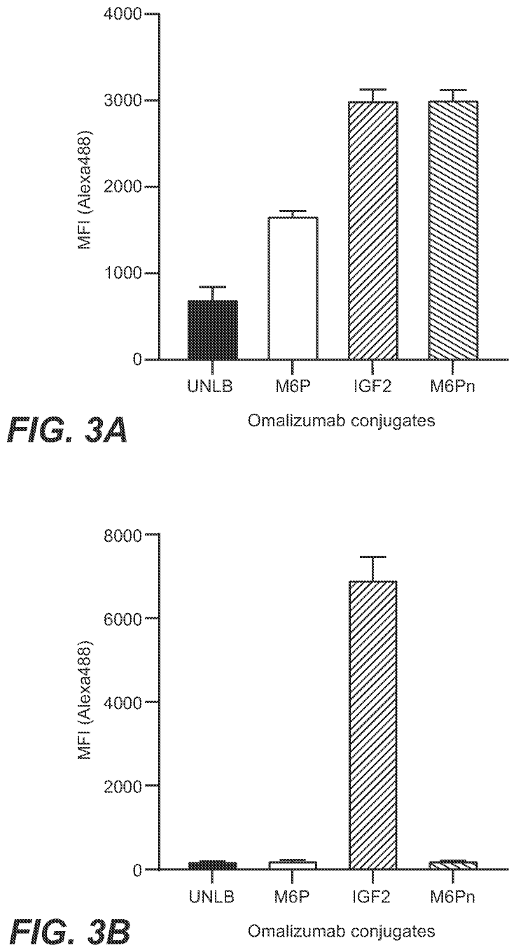

FIG. 3A shows a graph of MFI indicating extent of uptake for each composition in human Jurkat cells. FIG. 3B shows uptake in mouse C2C12 cells. FIG. 3C shows uptake in mouse fibroblasts. The cellular uptake is compared to omalizumab conjugates with glycan ligands for M6PR (mannose-6-phosphate ligand (M6P) or mannose-6-phosphonate analog (M6Pn)) and unconjugated omalizumab (UNLB).

Exemplary bifunctional compound (1) IGF-2-omalizumab was internalized to similar degree as M6Pn-omalizumab in Jurkat cells (FIG. 3A). Internalization of compound (1) IGF-2-omalizumab was also observed in the mouse myocyte cell line C2C12 (FIG. 3B) as well as primary mouse fibroblasts (FIG. 3C). No internalization of M6Pn-omalizumab or M6Pn-omalizumab conjugates was observed in either mouse cell type.

7.2.2. In Vitro Cell Uptake Assay with IR or IGF1R Receptor Inhibitors

The cell uptake assay is repeated in presence of IR and IGF1R blocking antibodies. Internalization of compound (1) IGF-2-omalizumab via IR and IGF1R cell surface receptors is reduced or eliminated in select cell types.

7.2.3. IGF2-Omalizumab is Internalized in K562^{M6PR-WT} Cells but not K562^{M6PR-KO} Cells

Omalizumab was conjugated to M6P, M6Pn, or IGF2 as described above, or left unconjugated (UNLB). The omalizumab compositions were then fluorescently labelled with Alexa 488 fluorescent dye as described above.

Uptake of the omalizumab-AF488 compositions was evaluated in K562 M6PR-wild type (WT) cells or K562 M6PR-knockout (KO) cells. Cells were incubated for 1 hour with composition and then analyzed by flow cytometry to measure mean fluorescent intensity (MFI) of cells.

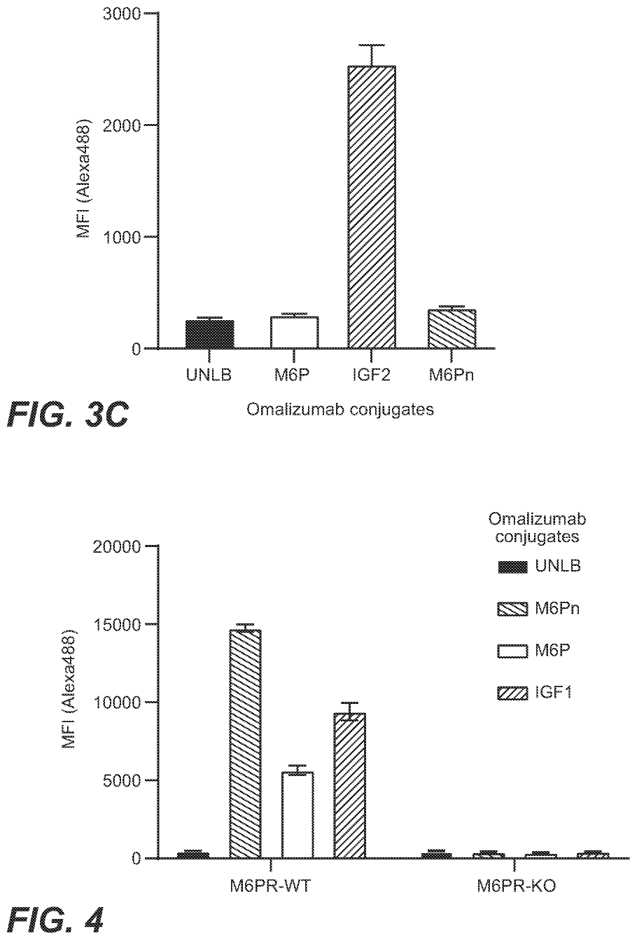

FIG. 4 shows the results of a cell uptake assay that illustrate that exemplary bifunctional compound (1) IGF-2-omalizumab is internalized in wild type K562 cells having M6PR but not M6PR-knockout (KO) K562 cells. Similar results were observed for omalizumab conjugates with glycan ligands for M6PR (M6P or M6Pn). UNLB is the control unconjugated omalizumab.

No internalization of exemplary bifunctional compound (1) IGF-2-omalizumab was observed in K562M6PR-KO cells, suggesting uptake in this cell line is mediated via M6PR. K562 cells do not express the receptors IR or IGF1R.

### 8. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A target-internalizing bifunctional compound, comprising:
an IGF-2 polypeptide; and
a target-binding moiety.

2. The compound of claim 1, wherein the target is a target protein and the bifunctional compound is capable of facilitating degradation of the target protein.

3. The compound of claim 1 or 2, wherein the target is an extracellular target protein.

4. The compound of claim 3, wherein the extracellular target protein is associated with a disease or disorder.

5. The compound of claim 3 or 4, wherein the extracellular target protein is a soluble target protein.

6. The compound of claim 5, wherein the extracellular target protein is an autoantibody.

7. The compound of claim 3 or 4, wherein the extracellular target protein is a cell membrane bound target protein.

8. The compound of any one of claims 1 to 7, wherein the target-binding moiety is an antibody or antibody fragment.

9. The compound of any one of claims 1 to 7, wherein the target-binding moiety is a peptide, a protein, or an aptamer.

10. The compound of any one of claims 1 to 7, wherein the target-binding moiety is a small molecule.

11. The compound of any one of claims 1 to 10, wherein the target-binding moiety has an affinity for the target of less than 1 μM.

12. The compound of any one of claims 1 to 11, wherein the IGF-2 polypeptide is a variant IGF-2 polypeptide having diminished or no affinity for the insulin receptor and/or IGFR1 as compared to naturally occurring human IGF-2 polypeptide.

13. The compound of any one of claims 1 to 11, wherein the IGF-2 polypeptide is a variant IGF-2 polypeptide having enhanced affinity for the CI-M6PR as compared to naturally occurring human IGF-2 polypeptide.

14. The compound of any one of claims 1 to 13, wherein the IGF-2 polypeptide comprises an amino acid sequence that is at least 80% identical to a sequence of Table 1.

15. The compound of any one of claims 1 to 14, wherein the IGF-2 polypeptide comprises a sequence selected from SEQ ID NO: 1-13.

16. The compound of claim 15, wherein the IGF-2 polypeptide consists essentially of a sequence of SEQ ID NO: 1-6.

17. The compound of any one of claims 1 to 16, wherein the IGF-2 polypeptide is covalently linked to the target-binding moiety via a linking moiety (e.g., a chemoselective ligation linker as described herein).

18. The compound of any one of claims 1 to 17, wherein the compound is of formula (I): 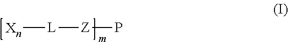
or a pharmaceutically acceptable salt thereof,
wherein:
X is the IGF-2 polypeptide;
n is 1 to 5 (e.g., n is 1 to 3, such as n is 1 or 2);
L is an optional linker;
Z is a residual linking moiety resulting from the attachment of Xn (or L, if present) to P via a chemoselective ligation group;
P is the target-binding moiety; and
m is 1 to 20 (e.g., m is 1 to 10, such as 2 to 10 or 2 to 6).

19. The compound of claim 18, wherein the linker (L) is linear.

20. The compound of claim 19, wherein the bifunctional compound comprises a ratio of IGF-2 polypeptide to target-binding moiety of about 1:1.

21. The compound of claim 19, wherein the bifunctional compound comprises two or more IGF-2 polypeptides linked to one target-binding moiety via linear linkers.

22. The compound of any one of claims 19-21, wherein the target-binding moiety is an antibody or antibody fragment.

23. The compound of claim 18, wherein the linker (L) is a branched linker.

24. The compound of claim 23, wherein the branched linker connects two or more IGF-2 polypeptides to one target-binding moiety.

25. The compound of claim 19, wherein the bifunctional compound comprises a ratio of IGF-2 polypeptide to target-binding moiety of about 2:1.

26. The compound of claim 25, wherein the target-binding moiety is an antibody or antibody fragment.

27. The compound of claim 22, wherein IGF-2 polypeptide is site-specifically covalently linked to the antibody or antibody fragment.

28. The compound of claim 27, wherein IGF-2 polypeptide is covalently linked to the antibody or antibody fragment via a site-specific cysteine modification on the antibody or antibody fragment (e.g., L443C) and a thiol-reactive chemoselective ligation group.

29. The compound of claim 22, wherein IGF-2 polypeptide is covalently linked to the antibody or antibody fragment via one or more lysine residues of the antibody or antibody fragment and an amine-reactive chemoselective ligation group.

30. The compound of claim 23, wherein the branched linker connects two or more target-binding moieties to one IGF-2 polypeptide.

31. The compound of claim 28, wherein the target-binding moiety is a small molecule.

32. The compound of any one of claims 1 to 17, wherein the bifunctional compound is a fusion protein comprising the IGF-2 polypeptide and a proteinaceous target-binding moiety.

33. The compound of claim 32, further comprising a spacer polypeptide (e.g., an intervening amino acid sequence) between the IGF-2 polypeptide and a proteinaceous target-binding moiety.

34. The compound of claim 32 or 33, wherein the proteinaceous target-binding moiety is an antibody or antibody fragment.

35. The compound of claim 34, wherein the IGF-2 polypeptide is fused to the antibody or antibody fragment via its C-terminal amino acid residue.

36. The compound of claim 34, wherein the IGF-2 polypeptide is fused to the antibody or antibody fragment via its N-terminal amino acid residue.

37. A method for internalizing an extracellular target in a cell, comprising:
contacting a biological system, comprising:
a cell having a M6PR cell surface receptor; and
an extracellular target;
with a bifunctional compound, comprising:
an IGF-2 polypeptide; and
a target-binding moiety;
to internalize the extracellular target in the cell.

38. The method of claim 37, wherein the extracellular target is an extracellular target protein associated with a disease or disorder.

39. The method of claim 36, wherein the extracellular target protein is degraded after internalization, thereby reducing levels of the extracellular target protein in the biological system.

40. The method of claim 38 or 39, wherein the target protein is a membrane bound protein.

41. The method of claim 38 or 39, wherein the target protein is a soluble target protein.

42. The method of any one of claims 37 to 41, wherein the biological system is a human subject.

43. The method of any one of claims 37 to 41, wherein the biological system is an in vitro cellular sample.

44. The method of any one of claims 37 to 43, wherein the bifunctional compound is according to any one of claims 2 to 36.

45. A method of treating a disease or disorder associated with a target protein, the method comprising:
administering to a subject in need thereof an effective amount of a bifunctional compound according to any one of claims 1 to 36, wherein the compound specifically binds the target protein and facilitates its internalization and degradation in cells of the subject.

46. The method of claim 45, wherein the disease or disorder is an inflammatory disease.

47. The method of claim 45, wherein the disease or disorder is an autoimmune disease.

48. The method of claim 45, wherein the disease or disorder is a cancer.
